(19) United States
(12) Patent Application Publication
(10) Pub. No.: US 2025/0281106 A1
(43) Pub. Date: Sep. 11, 2025

(54) NITRIC OXIDE BASED EVALUATION METHODS FOR INFECTION AND/OR SEPSIS AND DEVICES AND SYSTEMS FOR SAME

(71) Applicant: Vail Scientific, Inc., INVER GROVE HEIGHTS, MN (US)

(72) Inventors: Carter R. Anderson, Inver Grove Heights, MN (US); Russell L. Morris, Lindstrom, MN (US); Thomas Burke, Plymouth, MN (US); Clayton Anderson, Lakeville, MN (US)

(21) Appl. No.: 19/046,349

(22) Filed: Feb. 5, 2025

Related U.S. Application Data
(60) Provisional application No. 63/561,567, filed on Mar. 5, 2024.

(51) Int. Cl. A61B 5/00; A61B 5/0205; A61B 5/08; A61B 5/097; G01N 33/497

(52) U.S. Cl. CPC A61B 5/412; A61B 5/02055; A61B 5/082; A61B 5/097; A61B 5/7275; G01N 33/4975; A61B 2560/0223

(57) ABSTRACT

Disclosed herein are methods, devices, and systems useful in determining a sepsis risk of a subject non-invasively, wherein the methods, devices, and systems include measuring a nitric oxide concentration in the exhaled breath of the subject, and the determining has greater sensitivity than non-invasive methods lacking a nitric oxide concentration measurement.

### CROSS-REFERENCE TO RELATED APPLICATIONS

This application claims priority to U.S. Provisional Patent Application No. 63/561,567, filed Mar. 5, 2024, entitled “NITRIC OXIDE BASED EVALUATION METHODS FOR INFECTION AND/OR SEPSIS AND DEVICES AND SYSTEMS FOR SAME” which is incorporated by reference herein, in the entirety and for all purposes.

### FIELD

The described embodiments relate generally to methods, devices, and systems for determining sepsis and/or infection in a patient.

### BACKGROUND

Every year, severe sepsis strikes more than a million Americans. It is estimated that between 28 and 50 percent of these people die, more than the number from prostate cancer, breast cancer and AIDS combined. The Agency for Healthcare Research and Quality lists sepsis as the most expensive condition treated in U.S. Hospitals, costing more than $20 billion in 2011. Sepsis and septic shock are serious medical conditions caused by an overwhelming immune response to infection. Early diagnosis of sepsis has been shown to increase patient survival via appropriate treatment and decrease hospital stay/costs. Unfortunately, the currently available methods for diagnosing sepsis rely on detection of symptoms that only become evident after the infection has progressed to dangerous levels or require invasive testing methods. It has been estimated that as many as 80% of lives lost to sepsis could be saved if more rapid or accurate analysis was available.

Another problem associated with sepsis diagnosis results in potential for ‘over’ diagnosis, where patients are treated for sepsis despite not actually having it. Current methods for diagnosis of sepsis, using the quick Sequential Organ Failure Assessment (qSOFA) method, for example, can result in patients being administered antibiotics when they don't actually need them. The qSOFA score relies on assessing three different criterions-mentation or mental status, respiratory rate, and blood pressure. While the qSOFA score may be quickly calculated at the patient's bedside, it does not directly detect or assess the presence of microbes in the blood. A recent publication suggests that 56.7% of antibiotics administered to ER patients meeting qSOFA criteria are inappropriate.

Another proposed non-invasive screening tool for sepsis is a PRESEP score, which includes a calculation based on patients' heart rate, respiratory rate, body temperature, satO₂, and systolic blood pressure. While the PRESEP score is helpful, it likewise is deficient when it comes to identifying actual infection in a patient. For example, a PRESEP score may mis-identify a patient as having sepsis when they actually have heat stroke or a pulmonary embolism.

Another way to identify sepsis is with use of a scoring method, the SIRS score. The SIRS score is a 4-component method comprised of points awarded for 1) body temperature less than 36° C. or higher than 38° C. (1 point); 2) heart rate greater than 90 beats/min (1 point), 3) respiratory rate greater than 20 breaths/min (1 point) and 4) white blood cell count (WBC) greater than 12,000/mm³ or less than 4000 mm³ (1 point). Generally, a patient with a SIRS score of 2 points or higher is considered to have Sepsis 2 (as defined in Marik et al., J. Thoracic Disease, vol 9, no 4, Apr. 25, 20217). While most of these measurements are common and non-invasive, the WBC requires invasive sampling of the subject's blood.

Despite common use, the SIRS method has many inherent limitations. The blood sampling and test requirement can take several hours; there is a very high false positive rate; and it is sub-optimal for reliable identification of infection. The high false positive rate is particularly troublesome, in that there can be more false positives than true positives. Methods to identify false positives require using other invasive or slow tests, such as blood tests for procalcitonin or c-reactive protein (CRP); but these are invasive blood tests that will require even longer time periods for analysis. The false positive rate can also lead to unnecessary administration of antibiotics in patients that turn out to have a medical condition that is not sepsis.

Consequently, there exists a need for a fast, non-invasive test for sepsis that is better suited to identify infection.

### SUMMARY

Embodiments of the present invention are directed to a method for non-invasively determining a sepsis risk score in a human subject. In some examples, the method includes analyzing an exhaled breath, measuring nitric oxide in the breath to determine a measured nitric oxide concentration of the breath, obtaining at least one vital characteristic value of the subject selected from: a body temperature, a heart rate, or a respiratory rate, and assigning one point for each determined value if: the measured nitric oxide concentration is below 8 parts per billion (ppb) or above 12 ppb, the body temperature is below 36 degrees Celsius or above 38 degrees Celsius, the heart rate is greater than 90 beats per minute, or the respiratory rate is greater than 20 breaths per minute. The method further includes summing the assigned points to determine the subject's sepsis score, wherein if the sepsis score is greater than or equal to 2 the subject is prescribed systemic antibiotics.

A number of feature refinements and additional features are applicable in the first aspect and contemplated in light of the present disclosure. These feature refinements and additional features may be used individually or in any combination. As such, each of the following features that will be discussed may be, but are not required to be, used with any other feature combination of the first aspect.

In addition to the exemplary aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 results from flow rate correction of Nitric Oxide concentrations based on an exhaled flow rate.

FIG. 2 depicts a comparison between existing SIRS and the new SINO score for detecting disease.

FIG. 3A depicts a bar chart illustrating the incidence of September 2 as a function of triage based SINO score.

FIG. 3B illustrates data analysis of the SINO triage score for identifying bacterial and/or viral infections.

FIG. 4 illustrates ROC Curves for the data in Example 3.

FIG. 5A illustrates a bar chart illustrating the sensitivities of various measured characteristics of a patient.

FIG. 5B illustrates a table comparing the true positives, false negatives, and sensitivities of NO, CRP, procalcitonin, and white blood cells.

### DETAILED DESCRIPTION

The description that follows includes sample systems, methods, and apparatuses that embody various elements of the present disclosure. However, it should be understood that the described disclosure may be practiced in a variety of forms in addition to those described herein.

The following relates generally to methods, devices, and systems for determining bacterial infection, sepsis, and/or a sepsis risk score in a patient. Nitric oxide (NO) is a molecule in the blood that is dysregulated in sepsis. Because it has a very short half-life in blood, its measurement can be challenging and results from its measurement inaccurate. Applicants have determined that exhaled NO may be used alone or in conjunction with vital signs to predict bacterial infection and sepsis. As used herein, the term “infection” is used to indicate bacterial infection.

Infection and sepsis represent a spectrum of disease that lead to well described morbidity and death. Early diagnosis and treatment have been shown to improve outcomes. In some definitions, sepsis may be a severe or life-threatening condition, organ dysfunction, infection, or systemic inflammatory response causing or resulting from a dysregulated host or patient response to infection. In some examples, sepsis may occur with or without organ dysfunction. Sepsis may include or be alternatively referred to as septic shock.

Production of Nitric Oxide (NO) can be variable during sepsis. For example, NO production in sepsis is biphasic, although the timing and amplitude of NO production may vary. In some examples, a patient may exhibit early increases in NO production as infection or sepsis develops and decreases in NO production as sepsis evolves. A systemic inflammatory response may be triggered by increased production of NO from induced nitric oxide synthase. As sepsis progresses, however, NO levels drop, in some cases evolving into a ‘slow the inflammation’ phase. It has been hypothesized that the biphasic nature of a nitric oxide response may be related to polarization of M1 and M2 macrophages in response to microbial infection. In an M1 stage, increased production of nitric oxide may be associated with a microbial ‘killing’ phase, while inhibition of nitric oxide production may be associated with an M2 healing phase. Research suggests that this drop may be associated with increased mortality of a patient. Applicants show that exhaled NO levels that were either elevated or depressed, relative to published norms, are associated with sepsis.

Because NO has a very short half-life in blood, exhaled-breath-NO may provide a reliable means of measuring real-time values. As discussed herein, a tool for determining sepsis or a risk score of sepsis in a patient includes measuring exhaled breath NO in patients suspected to have an infection.

A sepsis risk score as described herein includes non-invasive measurements of vital signs in addition to the determination of exhaled NO level. In one example, the non-invasive sepsis risk score may be referred to as a SINO or VSNO score. The SINO score may include determination of one or more of a body temperature, heart rate, and respiratory rate combined with analysis of concentration of NO in exhaled breath. The measurements may result in obtaining a measured value or values for the characteristic. The measured value(s) are then compared to a threshold value for each characteristic, in some examples the threshold value may be a range, such as a threshold range value. If the measured value is above or below the threshold value, or within or outside the threshold range value, a point may be assigned for that characteristic. The sum of the points for each characteristic may define a summed sepsis risk score, or SINO or VSNO score. The presently disclosed SINO, VSNO, sepsis risk score has a higher sensitivity and specificity over existing diagnosis methods alone.

As used herein, “sensitivity” may refer to a test's ability to designate an individual with disease as positive. A highly sensitive test means that there are few false negative results, and thus fewer cases of disease are missed.

As used herein, “specificity” may refer to a test's ability to designate an individual who does not have a disease as negative.

Applicants show that the presently disclosed SINO score may have a sensitivity of about 0.9 and a specificity of about 0.5 for predicting sepsis. Applicants' results also showed a sensitivity of 0.8 and a specificity of 0.5 for predicting bacterial infection. Applied to a clinical setting, the disclosed SINO score would be immediately available to clinicians at the point of triage and would help identify patients who should receive expedited evaluation and care.

Identification of sepsis is both critical and challenging. Mortality rates of 24-32% have been recently described and mortality increases 7.6% for every hour antibiotic administration is delayed. The Surviving Sepsis Campaign International Guidelines recommends that administration of IV antimicrobials be initiated within one hour of recognition of sepsis or septic shock. Thus, sepsis should be identified as early as possible. Further, due to varying progressions of illness before seeking medical attention, accurately detecting the likelihood of sepsis on arrival to the emergency department (ED), or at the point of triage, may improve outcomes. This would then allow for immediate implementation of sepsis workup and treatment protocols.

Existing or standard screening methods lack specificity and/or sensitivity. For example an indicative SIRS (such as two or more SIRS vital signs having values outside of normal range, plus the suspicion of infection as identified by a triage nurse) may demonstrate a sensitivity of about or less than 40%, potentially missing over half of septic patients. Another existing method, quick Sequential Organ Failure Assessment (qSOFA) score (mental status, respiratory rate, and blood pressure) may demonstrate high specificity (82%), but likewise has a low sensitivity (46%) which again limits its utility as a triage screening tool. In an observational cohort study performed at one ED at an urban university hospital in Norway, qSOFA failed to identify two thirds of the patients presenting to an ED with severe sepsis.

Production of nitric oxide (NO) may be dysregulated in sepsis. NO is a signaling molecule which regulates several important processes involved in sepsis including vasodilation, neurotransmission, cardiac function, and immune system activation. NO production affects micro- and macrocirculation differently, leading to an overall mixed clinical effect. NO in the microcirculation may improve capillary exchange and prevent organ dysfunction (dilating and regulating vascular tone, preventing microvascular thrombosis, enhancing bacterial lysis by macrophages, inhibiting bacterial metabolism, and regulating leukocyte adhesion to endothelial cells), while NO in the macrocirculation causes a drop in systemic vascular resistance, hypotension, myocardial depression and shock.

While blood levels of NO cannot be accurately measured by blood draw due to the short half-life of the NO molecule, Applicants show that NO can be reliably measured in exhaled breath. NO concentration measurement in exhaled breath has been studied in asthmatic patients, where increased exhaled NO levels indicate increased inflammation of the airways. Reference ranges for fractional exhalation of NO (FeNO) in healthy adults have also been characterized. In a study of 2200 subjects, NO levels had a median value of 16 parts per billion (ppb), with a range of 2.4 to 199 ppb. Applicants disclose, herein, the first clinical study of NO levels in exhaled breath for differentiating sepsis or a likelihood of sepsis in septic and non-septic patients. For example, the study, disclosed herein, is the first to show a relationship between NO concentrations or levels in breath and a likelihood of sepsis in presenting sick or ill patients in an emergency room setting. The NO values or concentrations in exhaled breath may be affected by the NO analyzer used, measurement technique, exhalation rate, age, height, smoking, anti-inflammatory medications, and stress.

Applicants show that the exhaled levels of NO, alone or in combination with vital signs, can be used as a marker for the detection of infection and sepsis in patients with suspicion for infection. In some examples, the methods, devices, and systems disclosed herein provide measurements and/or calculations for accurately measuring exhaled-breath-NO in very ill patients, such as those with suspected sepsis, while compensating for the difficulty such patients may have in exhaling at a controlled rate. The presently disclosed compensation method can be used alone or in combination with patient biofeedback mechanisms and/or mechanical flow control mechanism used in a measuring instrument. Means to measure and/or control a flow rate may be developed in light of the teachings herein by those skilled in the art, and may include devices such as mass flow controllers.

Subjects and/or Patients

The disclosed methods, devices, and systems are useful in determining disease or conditions in various subjects. In some embodiments, the disclosed subject is a mammal, in particular a human. In many embodiments, the subject may or may not have suffer from or have a history of structural lung disease, asthma, chronic obstructive pulmonary disease (COPD), pulmonary fibrosis, be pregnant, using oxygen, using steroids, inhaled albuterol, corticosteroids, antibiotics, and/or require bi-level positive airway pressure (BiPAP). In many embodiments, the subject may or may not suffer from existing lung disease. In many embodiments, subjects with one or more existing condition may be analyzed using value or ranges of various components that may be different than those for subjects without existing conditions.

### Method of Determining a Sepsis Risk Score

Disclosed herein are methods of determining a subject's risk for having or developing a bacterial infection or sepsis. In many embodiments, the disclosed method may include analyzing a nitric oxide concentration in a subject's exhaled breath and one or more vital conditions of the subject. In many embodiments, the vital condition may be selected from heartrate (HR), respiratory rate (RR), and body temperature (temperature). In many embodiments, heart rate may be a value with units of heart beats per minute (bpm). In many embodiments, the respiratory rate may be a value with units of breaths per minute. In many embodiments, the body temperature may be a value with units of degrees Celsius or Fahrenheit. The various values may be measured electronically and/or manually. The measured values may be compared with a numerical range or a threshold, such as a minimum or maximum specific for that vital condition and, in some cases, for that subject. Medical professionals, such as a nurse, a nursing assistant, medical assistant, technicians, doctors, etc. may be able to measure and determine the necessary values.

A subject may be assigned a point if the measured value for nitric oxide concentration in an exhaled breath is inside or outside a threshold value range, or if the nitric oxide is above or below a threshold value. In one example, a point may be assigned if the nitric oxide concentration value is outside a defined threshold value range. In many embodiments, the threshold value range may be specific for a desired flow rate of breath. In many embodiments, the desired flow rate may be about 50 mL/sec, for example greater than about 40 mL/sec, 41 mL/sec, 42 mL/sec, 43 mL/sec, 44 mL/sec, 45 mL/sec, 46 mL/sec, 47 mL/sec, 48 mL/sec, 49 mL/sec, 50 mL/sec, 51 mL/sec, 52 mL/sec, 53 mL/sec, 54 mL/sec, 55 mL/sec, 56 mL/sec, 57 mL/sec, 58 mL/sec, or 59 mL/sec. In some embodiments the desired flow rate is between about 45 and 44 mL/sec. In many embodiments, the threshold range for nitric oxide concentration may be about 7 ppb to about 13 ppb. In many embodiments, the lower value may be less than about 10.0 ppb, 9.5 ppb, 9.0 ppb, 8.5 ppb, 8.0 ppb, 7.5 ppb, 7.0 ppb, 6.5 ppb, or 6.0 ppb, and greater than about 5.5 ppb, 6.0 ppb, 6.5 ppb, 7.0 ppb, 7.5 ppb, 8.0 ppb, 8.5 ppb, 9.0 ppb, or 9.5 ppb. In many embodiments, the upper value may be less than about 15.5 ppb, 15.0 ppb, 14.5 ppb, 14.0 ppb, 13.5 ppb, 13.0 ppb, 12.5 ppb, 12.0 ppb, 11.5 ppb, 11.0 ppb, or 10.5 ppb and greater than about 9.5 ppb, 10.0 ppb, 10.5 ppb, 11.0 ppb, 11.5 ppb, 12.0 ppb, 12.5 ppb, 13.0 ppb, 13.5 ppb, 14.0 ppb, 14.5 ppb, and 15.0 ppb. In some embodiments, the nitric oxide threshold range may vary depending upon one or more characteristics of the subject, for example, age, sex, existing conditions etc.

A subject may be assigned a point if the measured HR value is within or outside a value range, or below or above a threshold value. In one example, a point may be assigned if the measured HR value is above the threshold value. In many embodiments, the threshold value for HR is about 90. In some embodiments, HR threshold value may vary depending upon one or more characteristics of the subject, for example, age, sex, existing conditions etc. In some examples, the HR threshold value may correspond to a rate or value indicating tachycardia or a fast heart rate of a patient.

A subject may be assigned a point if the measured RR value is within or outside a value range, or below or above a threshold value. In one example, a point may be assigned if the measured RR value is above the threshold value. In many embodiments, the RR threshold value is about 20. In some embodiments, RR threshold value may vary depending upon one or more characteristics of the subject, for example, age, sex, existing conditions etc. In some examples, the RR threshold value may correspond to a rate or value indicating tachypnea or fast breathing of a patient.

A subject may be assigned a point if the measured body temperature value is within or outside a threshold range, or above or below a threshold value. In one example, a point may be assigned if the measured body temperature value is outside a threshold range. In many embodiments, the threshold range for temperature is 38 to 36° C. In some embodiments, temperature threshold range may vary depending upon one or more characteristics of the subject, for example, age, sex, existing conditions etc. In some examples, the threshold range for temperature may correspond to a temperature value indicating a hypothermia or hyperthermia in a patient.

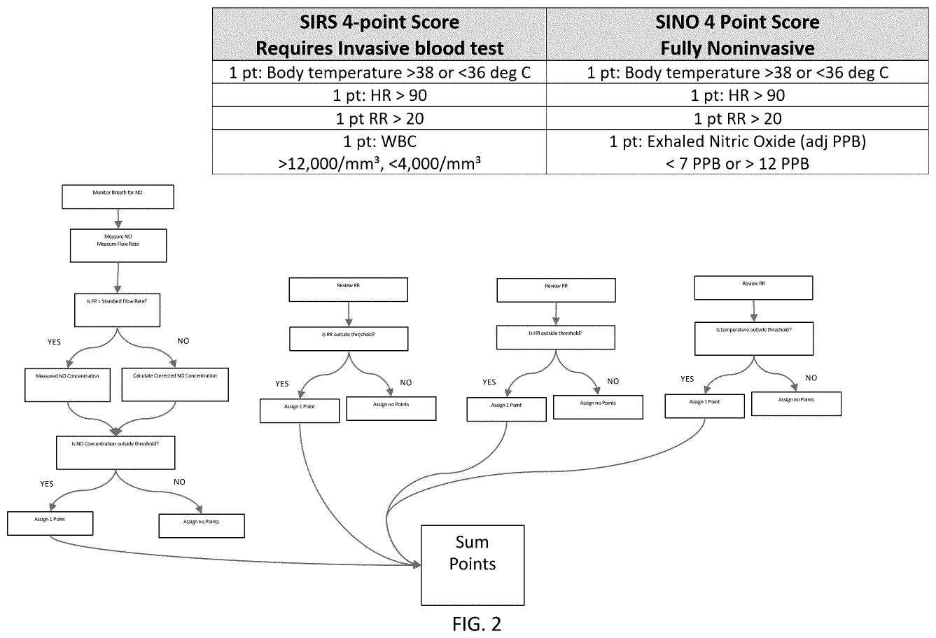

As disclosed herein, and depicted in FIG. 2, a sepsis risk score may be calculated for a subject wherein the sepsis risk score includes assessing NO concentration in exhaled breath, such as the disclosed VSNO or SINO scores, wherein the score is based on a summation of points based upon a comparison of measured values and threshold values for NO and one or more other vital conditions to create a summed risk score. For example, a subject may have a summed sepsis risk score of 0, 1, 2, 3, or 4. The summed sepsis risk score may be indicative of a subject's risk for having or developing a bacterial infection or sepsis. For example, if the summed risk score of the subject is above at or above limit, a treatment may be prescribed. In some examples, if the summed risk score is below a limit, a subject may be determined to not have sepsis. The treatment may include antibiotics, rest, additional observation, or the like as determined by medical professionals. In some examples, the limit may be 2 and a treatment may be prescribed if the summed sepsis risk score is 2 points or greater. In some examples, limit may be greater than 2, for example 3 or 4.

### Disease or Condition

Disclosed herein are methods of determining a subject's risk for having or developing an infection. In most embodiments, the infection is a bacterial infection, wherein one or more bacteria are present in the subject blood and/or urine.

The disclosed methods may be useful in detecting subjects having or at risk of developing sepsis. In some embodiments, sepsis may be Sepsis-2 or Sepsis-3 as determined or defined by one or more of the American College of Clinical Pharmacy (ACCP), the Society of Critical Care of Medicine (SCCM), or the ACCP/SCCM Consensus Conference Committee. Sepsis, as used herein, may refer to stage-2 sepsis (also referred to as “SEP2” or “Sep2” or “Sepsis 2”) or stage-3 sepsis.

The disclosed methods may aid in diagnosing bacterial infection and/or sepsis where other methods may fail. In one embodiment, triaged subjects may not meet SIRS criteria for bacterial infection or sepsis, but have a positive score when NO concentration is considered. In other embodiments, subjects may be diagnosed with a non-infection-based disease or condition, but have a positive score by the present method.

### Rapid Diagnosis

The disclosed methods, devices, and systems are useful in determining a subject's sepsis risk score without invasive monitoring or measurement. For clarity, measuring NO concentrations in exhaled breath, body temperature, heart rate, breath rate may be performed non-invasively. In contrast, measuring white blood counts, c-reactive protein concentration, and procalcitonin require invasive techniques, for example collecting a blood sample by venous puncture.

In many embodiments, the disclosed measurements may be performed during intake and/or triage of a subject. In some embodiments, the disclosed measurements may be performed in a medical facility, for example an emergency department, physician's office, bedside, point of care, ambulance, or during the triage process.

In many embodiments, the disclosed methods, devices, and systems are useful in obtaining early diagnosis of bacterial infection and sepsis. In these embodiments, early diagnosis may lead to earlier and/or expedited administration of care for example pharmaceutical administration, for example, antibiotics, intravenous fluids and other appropriate adjunctive care. In many cases, the disclosed methods, devices, and systems may aid in decreasing morbidity and mortality of a subject or a plurality of subjects. In many embodiments, the present methods, devices, and systems may decrease the time between collection of measurements and administration of care from about 24 hours to minutes or hours.

### Enhanced Sensitivity

The methods, devices, and systems disclosed herein are useful in identifying a subject at risk for having or developing an infection or sepsis with enhanced sensitivity. The disclosed method may be about 82% sensitive in detecting subjects with a bacterial infection. In many embodiments, the disclosed method may be greater than about 70%, 71%, 72%, 73%, 74%, 75%, 76%, 77%, 78%, 79%, 80%, 81%, 82%, 83%, 84%, 85%, 86%, 87%, 88%, 89%, or 90% and less than about 91%, 90%, 89%, 88%, 87%, 86%, 85%, 84%, 83%, 82%, 81%, 80%, 79%, 78%, 77%, 76%, 75%, 74%, 73%, 72%, or 71% sensitive. In many embodiments, the sensitivity of the present methods may be at least about 42 percentage points more sensitive than methods that do not include analysis of exhaled breath NO concentration—that is such methods may a sensitivity of only about 40% versus 82%—for example greater than about 30%, 35%, 40%, 45%, or 50% and less than about 60%, 55%, 50%, 45%, 40%, or 35%.

The disclosed methods may be about 89% sensitive in detecting subjects with a sepsis. In many embodiments, the disclosed method may be greater than about 75%, 76%, 77%, 78%, 79%, 80%, 81%, 82%, 83%, 84%, 85%, 86%, 87%, 88%, 89%, 90%, 91%, 92%, 99%, 94%, 95%, 96%, or 97% and less than about 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, or 76% sensitive. In many embodiments, the sensitivity of the presently disclosed methods may be at least about 35 percentage points more sensitive than methods that do not include analysis of exhaled breath NO concentration—that is such methods may a sensitivity of only about 54% versus 89%—for example greater than about 30%, 35%, 40%, 45%, or 50% and less than about 60%, 55%, 50%, 45%, 40%, or 35%.

The disclosed methods may be useful in identifying subjects that are not suffering from sepsis. In many embodiments, for example where the subject's sepsis risk score is less than about 2, the disclosed method may rule out sepsis with about 81% certainty.

### Site of Testing/Measuring/Analysis

In practice, NO testing and sepsis risk/SINO/VSNO score determination do not need to be limited to emergency department use. The test could be administered upstream from the emergency department including in clinics, ambulances, and nursing homes. An abnormal VSNO score may help more quickly identify patients becoming septic. This could be used in conjunction with other patient characteristics such as age and comorbidities to help triage appropriately.

### Breath Flow Rate

Fifty (48%) of enrolled patients were able to complete the breathalyzer at the goal rate of 50 ml/sec. For a healthy individual, blowing into a mouthpiece at a slow sustained rate for 10 seconds is straightforward. However, for the complex mix of emergency department patients with various illnesses, it is expected that some would find this to be challenging. For those that could not perform the sustained breath, an allowance was made for them to breathe at their preferred rate of exhalation, which was usually greater than 50 ml/sec. In all cases, adjusted corrected NO level was calculated for flow rate which was never exactly 50 ml/sec. Regardless of whether a patient was able to breathe at the goal rate or not, the corrected NO levels proved to have similar utility.

### No Concentration Analyzer Device

Disclosed herein is a device for measuring NO concentration in exhaled breath. In most embodiments, the disclosed device is also configured to measure flow rate of the exhaled breath. While in some cases the device, for example the device used in the presently disclosed study, was large (for example about the size of a desktop computer), other device dimensions are envisioned, including small portable devices that may analyze NO concentration and provide a value that is less reliant on the patient's exhalation rate.

Reference will now be made to Examples and accompanying drawings, which assist in illustrating various features of the present disclosure. The following description is presented for purposes of illustration and description. Furthermore, the description is not intended to limit the inventive aspects to the forms disclosed herein. Consequently, variations and modifications commensurate with the following teachings, and skill and knowledge of the relevant art, are within the scope of the present inventive aspects.

The term “about” or “approximately” means an acceptable error for a particular value as determined by one of ordinary skill in the art, which depends in part, on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

### EXAMPLES

### Example 1—Risk Score Calculation

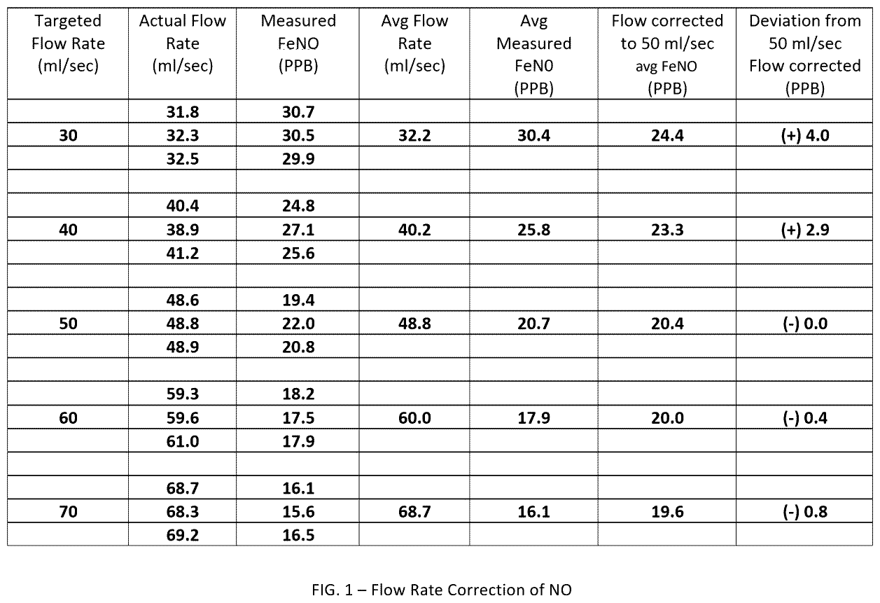

Turning to FIG. 1, in one example a risk score is calculated in part based on a concentration of nitric oxide (NO), wherein the NO concentration is adjusted or corrected to correspond to a standard flow rate based at least in part on the actual flow rate of a subject's exhaled breath. FIG. 1 illustrates results from an experiment where subjects were asked to exhale at a Targeted Flow Rate. The flow rate of the breath, along with NO concentration in the breath, were analyzed to determine an actual flow rate and a measured NO concentration over three separate breaths. The actual flow rates and measured NO concentrations were averaged.

Averaged NO concentrations may be adjusted to correspond to a standard flow rate. A standard flow rate may be a flow rate used to compare a measured NO concentrations to a threshold value or range. In many embodiments, the standard flow rate may be about 50 ml/sec (or mL/sec). In some embodiments, the standard flow rate may be about 30 mL/s to about 70 mL/sec, for example greater than about 30 mL/sec, 35 mL/sec, 40 mL/s, 41 mL/s, 42 mL/s, 43 mL/s, 44 mL/s, 45 mL/s, 46 mL/s, 47 mL/s, 48 mL/s, 49 mL/s, 50 mL/s, 51 mL/s, 52 mL/s, 53 mL/s, 54 mL/s, 55 mL/s, 56 mL/s, 57 mL/s, 58 mL/s, 59 mL/s, 60 mL/s, 61 mL/s, 62 mL/s, 63 mL/s, or 64 mL/s, and less than about 65 mL/s, 64 mL/s, 63 mL/s, 62 mL/s, 61 mL/s, 60 mL/s, 59 mL/s, 58 mL/s, 57 mL/s, 56 mL/s, 55 mL/s, 54 mL/s, 53 mL/s, 52 mL/s, 51 mL/s, 50 mL/s, 49 mL/s, 48 mL/s, 47 mL/s, 46 mL/s, 45 mL/s, 44 mL/s, 43 mL/s, 42 mL/s, 41 mL/s, 40 mL/s, or 35 mL/s.

The measured NO concentration of exhaled breath may be adjusted or corrected based on the flow rate of the exhaled breath to correspond to a standard flow rate. For example, a patient may be requested to target a flow rate based on their ability or strength, and the actual flow rate may be used to create a corrected NO concentration from the measured NO concentration.

An equation may be used for correcting the NO concentration to correspond to the standard flow rate (in this example 50 ml/sec). In many embodiments, the equation may be as follows:

$${NO}_{corrected} = 20 * {NO}_{at measured FR} / ( 208.6795 * F {R}^{- 0.5995} ) ;$$

where FR^{-0.5995} is the measured exhaled flow rate and NO at measured FR is the NO concentration measured at the FR and raised to the power of −0.5995.

In one example, a device or method utilizes 1) a flow rate measurement that is used in combination with 2) a mathematical correction equation to estimate a nitric oxide concentration in exhaled breath that would have been obtained if the patient exhaled at a nominal target rate such as, for example about 50 ml/sec.

The disclosed equation and correction method may be useful after measuring nitric oxide in exhaled breath of very ill patients. Such as those patients that may be experiencing sepsis (e.g. those with very low exhaled concentration of NO, such as less than 7 ppb nitric oxide at 50 ml/sec). The measured concentration at a flow rate deviating from a nominal rate can be mathematically adjusted to an estimated concentration that is then compared to a reference range expected at a nominal exhale rate so as to determine if a patient is at risk of having sepsis.

### Example 2—SINO Vs Existing Method

FIG. 2 illustrates an example comparison between existing SIRS, or systemic inflammatory response scores and the disclosed SINO score. The presently disclosed Nitric Oxide-based SINO score is similar to a SIRS score, except the WBC-based value in SIRS is substituted for an Exhaled Nitric Oxide concentration-based value. The WBC value requires invasive testing and analysis that delays diagnosis and treatment. Instead, the disclosed SINO score may be rapidly measured at a point of care. As depicted in the diagram below the table in FIG. 2, the disclosed SINO is calculated based on initial triage readings, or non-invasively readings obtained during patient intake.

The disclosed SINO score may include a body temperature component. In this component, a point or points may be associated with a body temperature value within and/or outside a given range or ranges of values, or above and/or below threshold values. For one example, 1 point, indicating a higher risk of or a sepsis condition, may be assigned for a body temperature value of a patient greater than about 38° C. or lower than about 36° C.

The disclosed SINO score may include a heart rate component. In this component, a point or points may be associated with a heart rate value within and/or outside a given range or ranges of values, or above and/or below threshold values. For one example, 1 point, indicating a higher risk of or a sepsis condition, may be assigned for a heart rate value greater than about 90 beats per minute (bpm).

The disclosed SINO score may include a respiratory rate component. In this component, a point or points may be associated with a respiratory rate value within and/or outside a given range or ranges of values, or above and/or below threshold values. For example, 1 point, indicating a higher risk of or a sepsis condition, may be assigned for a respiratory rate value greater than about 20 breaths per minute.

The disclosed SINO score includes an exhaled nitric oxide concentration component. In this component, a point or points may be associated with a nitric oxide concentration value within and/or outside a given range or ranges of values, or above and/or below threshold values. For example, 1 point, indicating a higher risk of or a sepsis condition, may be assigned for an exhaled nitric oxide concentration value below about 7 parts per billion (ppb) or greater than about 12 ppb. The concentration value may be the actual concentration value determined by a measurement apparatus or a corrected concentration value. In one example, where the measured flow rate of the exhaled breath is not equal to about 50 ml/sec, the measured concentration value may be modified by a multiplier so that it corresponds to a value taken at 50 ml/sec flow rate.

While example range values for the various components of the disclosed SINO score are disclosed herein, it is appreciated that alternative range values may be used in the assignment of a risk point. For example, the various ranges within the components may be adjusted based on a patient's age, height, weight, smoking, medications, stress, or existing medical conditions.

### Example 3—Study of NO and Sepsis Risk (SINO/VSNO Score Study)

104 patients (41 female) were enrolled in a study to examine the disclosed method. This study referred to the disclosed sepsis risk score, or SINO score, as a VSNO score (Vital Signs Nitric Oxide) in FIG. 6, the VSNO score may be based in part on the heart rate, respiratory rate, and body temperature of the patient. In this study, a point was assigned for an exhaled breath NO concentration value below about 8 parts per billion (ppb) or greater than about 13 ppb.

### Materials and Methods

The present study was conducted in the emergency departments (ED) of two large, urban tertiary care hospitals within one large healthcare network. One hospital has 509 inpatient beds and an ED census of 90,000 while the other hospital has 426 inpatient beds and an ED census of 60,000. Inclusion criteria were: emergency department patients age 18 years or older, blood cultures ordered, English speaking, ability to consent, breathing room air or oxygen by nasal cannula at the time of enrollment, ability to complete at least one exhalation breath test, and ability to have exhaled NO breath testing within six hours of ED arrival. Exclusion criteria were: history of structural lung disease (asthma, chronic obstructive pulmonary disease (COPD) or pulmonary fibrosis), pregnant, current use of home oxygen, use of inhaled albuterol or corticosteroids within the past month, current use of antibiotics at the time of presentation, and/or requirement for bi-level positive airway pressure (BiPAP) or oxygen by facemask at the time of enrollment. Patients were enrolled when research assistants were working and available to consent patients and collect data (24/7 at the larger hospital and 9 am-5 pm at the smaller). Fractional exhaled NO (FeNO hereafter reported as NO in units of parts per billion) was measured in the ED and all clinical data was collected from the electronic medical record. Patients were followed until death or until 48 hours after hospital discharge.

### Study Protocol.

ED patients were screened and identified by trained research assistants who were not blinded to the study aims but were unaware of patient diagnosis at the time of enrollment. Written informed consent was obtained. To collect NO concentration, each study subject exhaled through the device with each result being recorded separately. Up to three exhaled breaths were collected with the patient attempting to maintain a steady exhalation flow rate of 50 mL/sec with the device giving visual feedback on the flow rate. If more than one breath measurement was successful, the measurements were averaged. If the subject could not successfully exhale at a 50 mL/sec steady flow rate, they were instructed to exhale up to three times into the device at their preferred natural flow rate. Again, if more than one breath measurement was successful, the measurements were averaged. If the subject could not complete this task, they were excluded from the study. In all cases, the resultant NO levels were adjusted using the calculation described above in Example 1. 50 (48%) of the enrolled patients were able to exhale at the target rate of 50+/−5 mL/sec.

Clinical and laboratory data was collected from each subject's electronic medical record. Data was collected and managed using REDCap electronic data capture tools that consisted of clinical and diagnostic variables including, but not limited to, breathalyzer device data, clinical measurements, laboratory results, and readings from various imaging modalities. All exhaled NO data collection and all NO calculations were conducted by individuals who were unaware of patient infection/sepsis status.

In this Example, the cut-off or threshold values for abnormal levels of NO were: less than about 8 ppb or greater than about 13 ppb. One point was also assigned for each of the subject's RR, HR, or temperature outside of a stated threshold value or threshold range value. Again, a SINO/VSNO score of 2 points or more was considered a positive VSNO/SINO score.

| SIRS Score | VSNO Score |
|---|---|
| 1 pt: Body temperature >38 | 1 pt: Body temperature >38 |
| or <36° C. | or <36° C. |
| 1 pt: HR >90 beats per minute | 1 pt: HR >90 beats per minute |
| 1 pt: RR >20 breaths per minute | 1 pt: RR >20 breaths per minute |
| 1 pt: WBC >12,000/mm³ | 1 pt: Nitric Oxide (flow |
| or <4,000/mm³ | corrected ppb) <8 ppb or >13 ppb |

### Sepsis Diagnosis and Identification

Following patient discharge from the hospital (there were no in-hospital deaths), two physician investigators independently reviewed the patient chart to determine whether the patient had active infection (including source of infection when available), sepsis, or septic shock at the time of presentation to the ED. This determination was made utilizing the aforementioned collected data, chart notes, and following Sepsis-2 guidelines as very few patients met the sepsis definition by Sepsis-3 guidelines (Singer et al. The Third International Consensus Definitions for Sepsis and Septic Shock (Sepsis-3). JAMA 2016; 315(8):801-810). The few cases of disagreement were discussed until a consensus was obtained. Agreement was found in all cases. The investigators were blinded to the exhaled NO levels at the time of infection and sepsis determination.

### Outcome Measures

The goal of the study was to investigate whether measurement of exhaled NO, either alone or in combination with other clinical measurements, could:
Distinguish between enrolled ED patients who were ultimately diagnosed with sepsis and those in whom sepsis was not diagnosed (Primary Aim).
Distinguish between enrolled ED patients diagnosed with bacterial infection and patients not diagnosed with bacterial infection (Secondary Aim).

### Data Analysis

Demographics and clinical characteristics of the study sample were described using appropriate summary statistics (e.g. mean, standard deviation, median, range, frequencies). Analyses included comparing sensitivity and specificity for classifiers (NO alone, SIRS, VSNO) to predict disease (infection and sepsis). Optimized NO cut points, or threshold value or threshold range values, were calculated for the bounds of the normal range by maximizing Youden's J index to predict bacterial infection and sepsis while prioritizing higher sensitivity. The calculated cut points were less than 8 ppb or greater than 13 ppb. All statistical analyses were performed in SAS (Version 9: SAS, North Carolina, USA). Based on prior hospital data, a sepsis rate of 25% among those patients for whom blood cultures are ordered was anticipated. A total enrollment of 160 patients would result in 49 subjects anticipated to develop sepsis and 111 without sepsis, and should produce an AUC of 0.80 with a two-sided 95% confidence interval (0.719, 0.881).

### Results

Table 1 presents patient characteristics and triage data. Triage vital signs included: mean temperature 37.4° C., mean heart rate 108 beats per minute and mean respiratory rate 18.7 breaths per minute. The median exhaled NO level was 9.8 parts per billion (ppb) (IQR 5.6-17.0). 62 (60%) patients were diagnosed with bacterial infection and 54 (52%) patients were diagnosed with sepsis. Using optimized cut points of <8 or >13 ppb, the SINO/VSNO score demonstrated a sensitivity of 0.89 (95% CI: 0.77-0.96) and a specificity of 0.50 (95% CI: 0.36-0.64) for predicting sepsis. The score showed a sensitivity of 0.82 (95% CI: 0.70-0.91) and a specificity of 0.48 (95% CI: 0.32-0.64) for predicting bacterial infection.

TABLE 1
Patient Characteristics

| | Overall | | No sepsis | | Sepsis | |
|---|---|---|---|---|---|---|
| Total | 104 | | 50 | | 54 | |
| Age, M ± SD | 56.7 ± 18.1 | | 57.5 ± 18.6 | | 55.9 ± 17.8 | |
| Female, N (%) | 41 | (39.4) | 23 | (46.0) | 18 | (33.3) |
| Smoker, N (%) | 13 | (12.5) | 7 | (14.0) | 6 | (11.1) |
| Temperature, initial, M ± SD | 37.4 ± 1.0 | | 37.0 ± 0.9 | | 37.7 ± 1.0 | |
| HR, initial, M ± SD | 102 ± 18 | | 94 ± 19 | | 110 ± 14 | |
| SBP, initial, M ± SD | 129 ± 23 | | 128 ± 22 | | 129 ± 25 | |
| DBP, initial, M ± SD | 79 ± 16 | | 79 ± 16 | | 80 ± 17 | |
| RR, initial, M ± SD | 18.7 ± 3.2 | | 17.7 ± 3.3 | | 19.6 ± 2.9 | |
| Creatinine, initial, Median | 0.96 | (0.79-1.29) | 0.96 | (0.78-1.12) | 0.98 | (0.82-1.42) |
| (IQR) | | | | | | |
| Platelets, initial, Median (IQR) | 216 | (167-281) | 214 | (161-276) | 224 | (167-299) |
| Bilirubin, initial, Median (IQR) | 0.70 | (0.40-1.60) | 0.70 | (0.40-1.90) | 0.70 | (0.50-1.30) |
| Bacterial infection, N (%) | 62 | (59.6) | 15 | (30.0) | 47 | (87.0) |
| Viral infection, N (%) | 7 | (6.7) | 0 | (0.0) | 7 | (13.0) |
| Hospitalization, N (%) | 87 | (83.7) | 38 | (76.0) | 49 | (90.7) |
| Hospital total days length of | 3 | (2-6) | 2 | (1-5) | 4 | (3-7) |
| stay, Median (IQR) | | | | | | |
| Any vasopressor need, N (%) | 2 | (1.9) | 1 | (2.0) | 1 | (1.9) |
| ICU stay, N (%) | 3 | (2.9) | 2 | (4.0) | 1 | (1.9) |
| In-hospital death, N (%) | 0 | (0.0) | 0 | (0.0) | 0 | (0.0) |

As described above, a sepsis risk score based on points assigned for measured values of exhaled breath NO concentration and vital signs (VSNO/SINO) was created. Patients were given one point each for abnormalities of exhaled NO, Temp, heart rate (HR), and respiratory rate (RR). A summed sepsis risk score of two or more was considered positive or abnormal. Test and score characteristics are reported in Table 2.

TABLE 2
Test characteristics and score characteristics

| | Overall | | No sepsis | | Sepsis | |
|---|---|---|---|---|---|---|
| Total | 104 | | 50 | | 54 | |
| NO, Median (IQR) | 9.8 | (5.6-17.0) | 9.6 | (3.9-15.2) | 10.6 | (6.0-17.1) |
| WBC, Median (IQR) | 10.7 | (7.7-15.4) | 9.1 | (5.7-13.3) | 12.2 | (9.5-16.9) |
| SIRS using triage vitals (2+), N (%) | 60 | (57.7) | 15 | (30.0) | 45 | (83.3) |
| SINO/VSNO score using triage vitals | 73 | (70.2) | 25 | (50.0) | 48 | (88.9) |
| (2+), N (%) | | | | | | |
| Procalcitonin, initial, Median (IQR) | 0.20 | (0.09-0.78) | 0.12 | (0.07-0.22) | 0.50 | (0.15-5.97) |
| C-reactive protein, initial, Median | 8.4 | (4.6-17.8) | 6.1 | (2.5-17.6) | 10.1 | (5.9-18.5) |

(IQR)

The SINO/VSNO score performed well at detecting bacterial infection and sepsis. In a clinical setting, this disclosed sepsis risk score would be immediately available to clinicians at the point of triage and would help to identify patients who should receive expedited evaluation and care.

### VSNO Score and Sepsis

A positive VSNO score demonstrated a sensitivity of 0.89 (95% CI: 0.77-0.96) and a specificity of 0.50 (95% CI: 0.36-0.64) for predicting sepsis. The PPV was 0.66 (95% CI: 0.54-0.76) with a NPV of 0.81 (95% CI: 0.63-0.93). Comparatively, meeting SIRS criteria by the initial triage vital signs alone had a sensitivity of 0.54 (95% CI: 0.40-0.67), with a specificity of 0.80 (95% CI: 0.66-0.90)—Table 3.

TABLE 3
Score diagnostic performance for detecting sepsis.

| | | SIRS score using triage |
|---|---|---|
| | VSNO | vitals only (no WBC) |
| Sensitivity (95% CI) | 0.89 (0.77-0.96) | 0.54 (0.40-0.67) |
| Specificity (95% CI) | 0.50 (0.36-0.64) | 0.80 (0.66-0.90) |
| PPV (95% CI) | 0.66 (0.54-0.76) | 0.74 (0.58-0.87) |
| NPV (95% CI) | 0.81 (0.63-0.93) | 0.62 (0.49-0.73) |
| AUC (95% CI) | 0.69 (0.61-0.78) | 0.67 (0.58-0.76) |

### VSNO Score and Bacterial Infection

A positive VSNO score showed a sensitivity of 0.82 (95% CI: 0.70-0.91) and a specificity of 0.48 (95% CI: 0.32-0.64) for predicting bacterial infection. The PPV was 0.70 (95% CI: 0.58-0.80) and the NPV was 0.65 (95% CI: 0.45-0.81)—Table 4. SIRS scores using triage vital signs produced a sensitivity of 0.40 (95% CI: 0.28-0.54) and a specificity of 0.67 (95% CI: 0.50-0.80)

TABLE 4
Score diagnostic performance for detecting bacterial infection.

| | | SIRS score using triage |
|---|---|---|
| | VSNO | vitals only (no WBC) |
| Sensitivity (95% CI) | 0.82 (0.70-0.91) | 0.40 (0.28-0.54) |
| Specificity (95% CI) | 0.48 (0.32-0.64) | 0.67 (0.50-0.80) |
| PPV (95% CI) | 0.70 (0.58-0.80) | 0.64 (0.47-0.79) |
| NPV (95% CI) | 0.65 (0.45-0.81) | 0.43 (0.31-0.56) |
| AUC (95% CI) | 0.65 (0.56-0.74) | 0.53 (0.44-0.63) |

These studies show that nitric oxide, as part of a clinical score including triage vital signs, demonstrated reasonable accuracy in detecting bacterial infection and sepsis in emergency department patients presenting with concern for infection.

Disclosed herein is a unique sepsis risk score (SINO/VSNO) that includes components of triage vital signs with exhaled breath NO concentration while excluding invasive diagnostic results. This score showed good test characteristics for evaluating patients for bacterial infection and sepsis. In particular, the score showed a sensitivity of 82% for detecting any bacterial infection and 89% for detecting sepsis.

Compared with vital signs alone which are typically used early in a patient's ED triage evaluation for sepsis, the use of exhaled NO levels improved sensitivity from 54% to 89%. Additionally, the NPV indicates that a summed sepsis risk score less than 2 can rule out sepsis with 81% certainty. In ED triage, this could lower the triage acuity level of a patient with a presenting complaint of fever but no other abnormality of vital signs or exhaled NO level. In contrast to other traditionally used biomarkers such as WBC, CRP and procalcitonin, the measured or corrected NO value can be obtained non-invasively and can produce results immediately. Indeed, all of the components of the disclosed sepsis risk score can be easily obtained by a nurse or nursing assistant during the triage process.

The high sensitivity of the disclosed sepsis risk score as contrasted with vital signs alone may be due to many septic patients not presenting with abnormal HR, RR, or temperature values. One benefit of NO screening is finding those patients who may appear well (i.e. only one point from abnormal HR, RR, or temperature value(s)) but in whom sepsis is developing (the most feared of all ED patients; sick patients masquerading as well). In particular, one subject from the present study presented through triage but did not meet SIRS criteria on arrival and was initially discharged from the ED with a diagnosis of a UTI. This patient did have a positive disclosed sepsis risk score on that day (based on their heart rate and NO level), and ultimately, they were called to return to the ED the following day when both their urine and blood cultures were positive for MRSA. The positive disclosed sepsis risk score could have allowed this patient early intervention.

Another potential benefit of NO data may be to support or refute a diagnosis of sepsis in the common situation when the diagnosis is unclear. As an example, one study patient was ill-appearing on presentation and was admitted for alcohol withdrawal with a heart rate of 130. They were deemed unlikely to have an infection based on their workup, and antibiotics were not started. The following day, their blood cultures grew MRSA, and they were ultimately treated for sepsis secondary to MRSA bacteremia. Their NO value on arrival had been markedly low (<1.0 ppb) and the disclosed sepsis risk score was abnormal.

In practice, NO testing with sepsis risk score determination do not need to be limited to emergency department use. The test could be administered upstream from the emergency department including in clinics, ambulances, and nursing homes. An abnormal score may help more quickly identify patients becoming septic. This could be used in conjunction with other patient characteristics such as age and comorbidities to help triage appropriately.

As noted above, Fifty (48%) enrolled patients were able to complete the breathalyzer at the goal rate of 50 ml/sec+/−5 mL/sec. For a healthy individual, blowing into a mouthpiece at a slow sustained rate for 10 seconds is straightforward. However, for the complex mix of emergency department patients with various illnesses, it is expected that some would find this to be challenging. As noted above, for those patients that could not perform the sustained breath, they were asked to exhale at their preferred rate (was usually greater than 50 ml/sec). In these cases, the measured NO concentration value was corrected to correlate with a standard flow rate. Corrected NO levels proved to have similar utility.

### Example 4

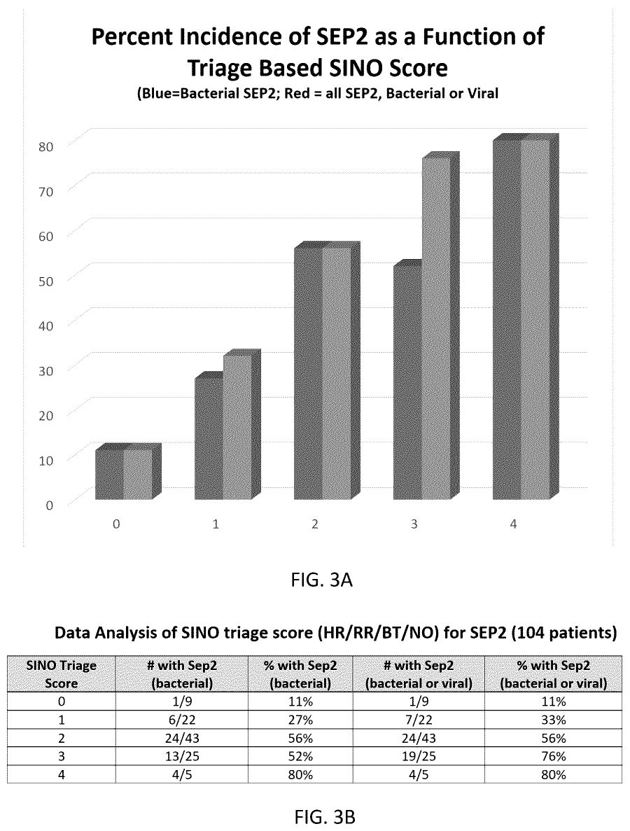

Turning to FIGS. 3A and 3B, the chart and table, respectively, illustrate example experimental results demonstrating the use of the currently disclosed sepsis risk score to determine severe or stage-2 sepsis (“SEP2” or “Sep2” or “Sepsis 2”). In this example, the disclosed sepsis risk score may be referred to as SINO, while other embodiments may refer to “VSNO” and/or the “disclosed sepsis risk score” to indicate the presently disclosed method for non-invasively determining a subject's risk of having or developing sepsis 2. In some embodiments, the SINO score may be useful in identifying subjects with the presence of bacterial or viral-based Sep2. FIG. 3A is a bar graph showing the percentage of patients positive for SEP2 with a given SINO score. For example, about 10 percent of patients with a “0” SINO score were positive for SEP2. The table in FIG. 3B shows actual values—i.e. 1 of 9 patients with a “0” SINO score, 11%, were determined to have SEP2.

Higher SINO scores determined by the disclosed methods, as depicted in FIGS. 3A and 3B, correlate with higher incidence of Sep2. Patients with a summed SINO Score of 3 points or higher have about a 76% likelihood of having Sepsis 2 (bacterial or viral). Patients with a summed SINO Score of 2 points or higher have approximately 56% likelihood of having severe sepsis. Patients with a summed SINO score of 4 points have about an 80% likelihood of having severe sepsis. Accordingly, as a majority of patients (i.e. greater than about 50%) with a SINO score of 2 points or greater may have an infection or sepsis, a summed SINO score of 2 points may be a limit for determining if a patient should be treated for Sepsis 2. In some embodiments, the limit may be higher or lower than 2 points.

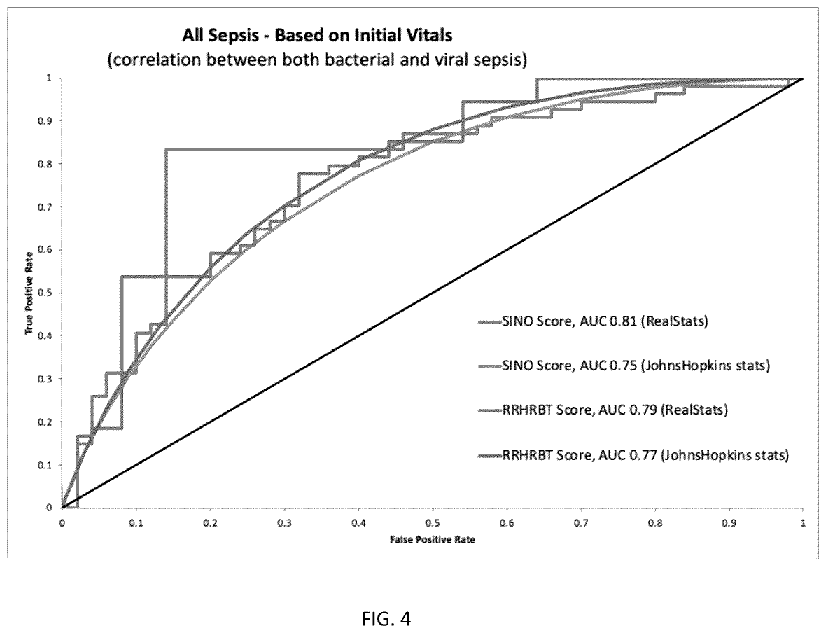

FIG. 4 is a receiver operating characteristic curve, or ROC curve for the data comparing the ability of non-invasive screening characteristic values such as heart rate value, respiratory rate value, and body temperature value to determine Sep2 in a patient without (HR/RR/BT-only) and with (i.e. SINO score) inclusion of an exhaled breath nitric oxide concentration value component. A ROC curve is a graphical illustration of the performance of a binary classifier model at varying threshold values-a straight line indicates poor predictive value, while curves above that line indicate improved predictive value with greater deviations correlating with greater predictive value.

The ROC curves generated from the present data indicate that both screening methods may be acceptable. The area under the curves (AUCs) are all over 0.7. However, the curve generated from the SINO score data comes with a much improved sensitivity to identify patients at risk of sepsis. While the data generated with points from HR/RR/BT-only has an acceptable ROC AUC, it misses nearly half of patients at risk of having sepsis. Accordingly, use of exhaled breath NO (SINO) scores are better at “ruling in” likelihood of sepsis:
SINO scores will catch septic patients that heart rate (HR)/respiratory rate (RR) value/body temperature (BT) value misses. For example, in this study, 18 patients would have been missed by analyzing HR/RR/BT-only that were appropriately caught by a SINO score. A HR/RR/BT-only scores misses almost as many patients as it catches. With sepsis being a high mortality risk, the consequence of missing patients is worse than the consequence of having a higher rate of false positives.
SINO scores also have increasing positive predictive value with increasing score. Scores of 3 or 4 points have a positive predictive value for sepsis of 77%.
SINO scores are better at “ruling out” likelihood of sepsis. For example, SINO Scores offer a better Negative Predictive Value, a SINO score of 0-1 has a negative predictive value of 77.4% (likelihood that they won't have sepsis) while a HR/RR/BT-only score of 0-1 has a negative predictive value of 62.1%.

Using Logistic Regression provides the same results and observations. While the AUCs of curves generated using either method are similar (seen in the FIG. 4 ROC plot), the reasons are very different. In the case of the SINO score data, the sensitivity is 87%, as stated previously, correctly identifying 47 out of 54 patients at the initial triage stage and prior to any blood work. The HR/RR/BT-only score properly identified only 54% or 29 out of the same 54 patients. Despite this significant difference, the ROC AUCs are similar because, compared to the SINO score, the HR/RR/BT-only score was more effective at ruling out, or identifying patients that did not later receive a Sep2 diagnosis. Given the need to rapidly and accurately screen patients likely to have sepsis, greater sensitivity is useful. Combining NO with the other vitals (the SINO test score) at the initial triage point is far superior to a test score lacking NO.

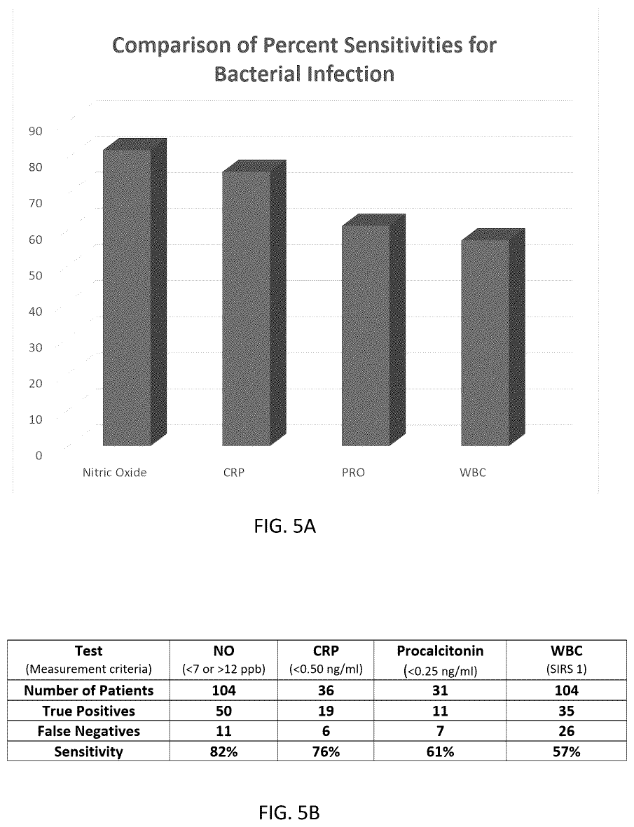

Turning to FIGS. 5A and 5B, experimental results are illustrated where nitric oxide concentration values are included as a screening factor or point in the SINO score, compared to existing and invasive methods. For example, an NO value above or below the threshold (see FIG. 2) is compared to a C-reactive protein (CRP) concentration (in ng/ml), procalcitonin (in ng/ml), and white blood cell counts (as may be used in existing SIRS scores). A premise to substituting NO for WBC lies in its comparative ability to help identify bacterial infection, as illustrated by the results in FIG. 5A and FIG. 5B.

In a screening test for a patient at risk for sepsis, sensitivity is an important measure. Sensitivity is the percentage of positive patients that were appropriately identified in the test. Low sensitivities mean patients at risk will be missed.

The true positives (TP) were determined by comparing whether the measured condition correctly identified a bacterial infection. For example, TP=true positives vs. infection. False negatives (FN) were determined by comparing incorrectly identified negatives where bacterial infection was present. For example, FN=false negatives vs infection. The sensitivity was determined by the number of true positives divided by the total number of determined bacterial infections (e.g. true positives and false negatives). For example, sensitivity=TP/[TP+FN]×100%.

Other examples and implementations are within the scope and spirit of the disclosure and appended claims. For example, features implementing functions may also be physically located at various positions, including being distributed such that portions of functions are implemented at different physical locations. Also, as used herein, including in the claims, “or” as used in a list of items prefaced by “at least one of” indicates a disjunctive list such that, for example, a list of “at least one of A, B, or C” means A or B or C or AB or AC or BC or ABC (i.e., A and Band C). Further, the term “exemplary” does not mean that the described example is preferred or better than other examples.

The foregoing description, for purposes of explanation, uses specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A method for non-invasively determining a sepsis risk score in a human subject comprising:
analyzing an exhaled breath;
measuring nitric oxide in the breath to determine a measured nitric oxide concentration of the breath;
obtaining at least one vital characteristic value of the subject selected from:
a body temperature,
a heart rate, or
a respiratory rate;
assigning one point for each determined value if:
the measured nitric oxide concentration is below 8 parts per billion (ppb) or above 12 ppb,
the body temperature is below 36 degrees Celsius or above 38 degrees Celsius,
the heart rate is greater than 90 beats per minute, or
the respiratory rate is greater than 20 breaths per minute, and
summing the assigned points to determine the subject's sepsis score, wherein if the sepsis score is greater than or equal to 2 the subject is prescribed systemic antibiotics.

2. The method of claim 1, wherein the measured nitric oxide concentration is calculated by:
measuring a flow rate (FR) for the exhaled breath;
employing a correction factor to adjust the nitric oxide concentration to a standard flow rate.

3. The method of claim 2, wherein the employing includes multiplying the measured nitric oxide concentration and a correction factor, wherein the correction factor is:
20/(208.6795*FR^{-0.5995}), wherein FR is the measured flow rate of the exhaled breath.

4. The method of claim 2, where the measuring nitric oxide is performed with a device comprising a flow control circuit.

5. The method of claim 3, where the measuring nitric oxide is performed with a device comprising a flow control circuit.

6. The method of claim 2, where the measuring nitric oxide is performed with a device comprising a circuit and a mass flow controller.

7. The method of claim 3, where the measuring nitric oxide is performed with a device comprising a circuit and a mass flow controller.

8. The method of claim 4, where the measuring nitric oxide is performed with a device comprising a circuit and a mass flow controller.

9. The method of claim 5, where the measuring nitric oxide is performed with a device comprising a circuit and a mass flow controller.
